(19) United States
(12) Patent Application Publication
(10) Pub. No.: US 2008/0014607 A1
(43) Pub. Date: Jan. 17, 2008

(54) ATP-METRY BASED ON INTRACELLULAR ADENYL NUCLEOTIDES FOR DETECTING AND COUNTING CELLS, USE AND IMPLEMENTING METHOD FOR DETERMINING BACTERIA IN PARTICULAR DEVOID OF ATP

(75) Inventor: Dominique CHAMPIAT, Les Matelles (FR)

Correspondence Address: FOLEY & LARDNER LLP, 975 PAGE MILL ROAD, PALO ALTO, CA, 94304, US

(73) Assignee: Testlife Technologies

(21) Appl. No.: 11/737,078

(22) Filed: Apr. 18, 2007

Related U.S. Application Data
(63) Continuation of application No. PCT/FR05/02595, filed on Oct. 19, 2005.

(30) Foreign Application Priority Data
Oct. 19, 2004 (FR) 0411084

(51) Int. Cl. C12Q 1/06

(52) U.S. Cl. 435/39

(57) ABSTRACT

The invention concerns the use of bioluminescence dependent on the reaction (1): luciferin+ATP+O₂+Mg²⁺+luciferase→oxyluciferin+photons for detecting and counting living cells of a given species potentially present in a liquid sample, said use being characterized in that it consists in measuring the total free intracellular adenyl nucleotides (AN) content, expressed in ATP form, of living cells of a given non-viral species, taking into account the fact that the sum of free intracellular ATP, ADP and AMP of said family is constant according to the relationship (2): [AN]=[ATP]+[ADP]+[AMP]=Cte after transforming the free intracellular ATP, ADP and AMP by using myokinase and pyruvate kinase, said measurement being performed (i) without adding ATP and (ii) after adding a known amount of ATP. The invention also concerns a method for detecting and counting cells by ATP-metry.

### FIELD OF THE INVENTION

The present invention relates to a novel ATP-metry technique based on free intracellular adenyl nucleotides (ANs), for detecting and counting cells. It also relates to the use of this novel technique and to an implementing method for determining bacteria, in particular those which are devoid of ATP.

### PRIOR ART

It is known that ATP-metry, which is based on the reaction:
luciferin+ATP+O₂+Mg²⁺+luciferase→oxyluciferin+photons, (1)

makes it possible to effectively measure the ATP content of a medium. This reaction is specific for ATP, irrespective of the luciferin (substrate)/luciferase (enzyme) system used. It makes it possible to distinguish between dead cells (devoid of ATP) and living cells when the latter contain ATP.

In the past, it was believed (in vain) that knowledge of the content of intracellular ATP originating from the lysis of bacteria of the same species could make it possible to detect and count the population (i.e. number of strains per unit volume) of said bacteria. To this effect, see the publications U.S. Pat. No. 6,200,767 B, EP 1333097 A, U.S. Pat. No. 6,465,201 B, H. Stender et al., Journal of Microbiological Methods, 2001;46:69-75, and Lee J-Y, et al., Luminescence 2004;19:31-36.

The methods described in these publications are effective for studying bacteria that come from the same collection culture and that are all at the same state of development (i.e. bacteria which are not dormant and which all contain the same ATP content). On the other hand, they are ineffective with respect to virtually all other bacteria that are encountered, in particular in nature, i.e. especially (i) bacteria that do not contain any ATP (this is the case of bacteria which are in the form of spores, i.e. dormant), and (ii) bacteria which are in different states of development and which, as a result, do not each have the same ATP content.

Now, it is known that, within the same species or variety of cells, the content of total free intracellular ANs is constant, considering relationship (2):
[AN]=[ATP]+[ADP]+[AMP]=Ct (2)

To this effect, see the article by D. Champiat et al., Luminescence, 2001;16:193-198, where it proposed, firstly, to convert the AMP and, respectively, the ADP to ATP by means of pyruvate kinase and, respectively, myokinase, and, secondly, to measure the light emitted (in RLU, i.e. in relative light units) without the addition and then after the addition of a further 10 μl of ATP.

Moreover, a technique for evaluating the presence of contaminants, such as microorganisms (in particular bacteria), in an aqueous sample originating from seawater, from drinking water or from a food, is known from said article by D. Champiat. This technique comprises bringing a sample to be tested into contact with the luciferin+luciferase combination so as to measure the light emitted (i.e. to measure, with amplification, the photons produced) by the abovementioned reaction (1) in the presence of microorganisms that release ATP, ADP and/or AMP, firstly, with conversion of the ADP and AMP to ATP and, secondly, with and without the further addition of ATP. Said article neither describes not suggests that this technique is applicable to the detection and counting of cells containing at least one of the three ANs. In fact, this article is aimed merely at measuring the emitted light, in RLU, without thinking it possible to correlate the values obtained with the content of total free intracellular ANs and then with the number of cells having supplied said total free intracellular ANs, or suggesting this correlation.

### OBJECTIVE OF THE INVENTION

There exists a need as regards a technique for detecting and counting cells, in particular bacteria, molds and microscopic algae, rapidly, effectively and in a manner that is much less expensive than the EIA, RIA, FIA and PCR methods currently recommended.

This need becomes acutely apparent when it is desired to count microorganisms devoid of ATP or AN.

It is therefore proposed to provide a novel technical solution implementing ATP-metry relating to all total free intracellular ANs, expressed in the form of ATP, in order to meet this need.

### SUBJECT OF THE INVENTION

The present invention makes it possible to meet said need for all cells, with the exclusion of viruses which do not contain any ATP (in fact, viruses, which do not have any ANs, use the ATP of the cells that they infect, in order to develop).

According to a first aspect of the invention, use of bioluminescence according to reaction (1):
luciferin+ATP+O₂+Mg²⁺+luciferase→oxyluciferin+photons, (1)

is provided for detecting and counting living cells of a given species that may be present in a liquid sample, said use being characterized in that it implements measuring the content of total free intracellular adenyl nucleotides (ANs) expressed in the form of ATP, of living cells of a given nonviral species, taking into account the fact that the sum of free intracellular ATP, ADP and AMP of said family is constant according to relationship (2):
[AN]=[ATP]+[ADP]+[AMP]=Ct, (2)

after having converted the free intracellular ADP and AMP to ATP by means of myokinase and pyruvate kinase, said measurement being carried out (i) without the addition of ATP and (ii) after the addition of a known amount of ATP.

According to a second aspect of the invention, a method is provided for detecting and counting the cells of a given nonviral species that are liable to be present in a liquid sample (S), in particular bacteria, by means of a method of bioluminescence according to reaction (1):
luciferin+ATP+O₂+Mg²⁺+luciferase→oxyluciferin+photons, (1)

said method, which is based on the fact that, for a living cell of a given nonviral species, the sum of the intracellular adenyl nucleotides (ANs) is constant according to relationship (2):
[AN]=[ATP]+[ADP]+[AMP]=Ct, (2)

being characterized in that it comprises the following steps consisting in:

(1°) isolating and concentrating the cells of said given species that are liable to be present in the sample (S), after having to remove the extracellular ANs that may be contained in said sample;

(2°) lysing the wall of the cells;

(3°) treating the resulting liquid medium in order to convert the intracellular ADP and AMP which it contains, and which originate from said cells, to ATP;

(4°) introducing, into the medium resulting from step (3°), a luciferin and a luciferase, first (i) without the addition of ATP, and then (ii) after the addition of a known amount of ATP;

(5°) measuring the amplified signal of the light emitted by reaction (1) without the addition of ATP, and then after the addition of a known amount of ATP;
and

(6°) determining the content of total free intracellular ANs in the form of ATP, by comparison with the reproduction of steps (1°) to (5°) with a known population of said cells.

According to another aspect of the invention, firstly, a use of said method for counting bacteria in the form of spores, which, as a result, are devoid of ATP, and, secondly, an assay kit for implementing said method are provided.

Said assay kit is characterized in that it comprises the firefly luciferin/luciferase combination, ATP for the metered addition, myokinase, pyruvate kinase and, where appropriate, pyruvate orthophosphate dikinase and/or adenosine phosphate deaminase.

Abbreviations

For convenience, the list of abbreviations and acronyms used in the present invention has been provided below.

ADP adenosine diphosphate,

AMP adenosine monophosphate,

AN adenyl nucleotide (other nomenclature that can be used: adenosine nucleotide), the collection of ANs comprises herein ATP, ADP and AMP,

ATP adenosine triphosphate,

cAMP cyclic adenosine monophosphate,

GDP guanosine diphosphate,

GTP guanosine triphosphate,

RLU relative light unit.

### DETAILED DESCRIPTION OF THE INVENTION

The sample (S), which is an aqueous or organic liquid composition, is advantageously an aqueous composition, and the cells to be tested are advantageously bacteria. This sample (S) comes from a gas sample taken [in particular by sparging], a solid sample taken [in particular by contact, dissolution or dispersion] or a liquid sample taken [in particular by extraction, dissolution or emulsion] by means of a liquid which is advantageously aqueous.

The abovementioned reaction (1) provides oxyluciferin, photons, AMP and one or more phosphates, mainly pyrophosphate. It is characteristic of living matter since the intracellular ATP released into the reaction medium does not have a long lifetime. It is specific for ATP, the luciferin and luciferase being at an optimal concentration, and the number of photons emitted once these three substances are present together, is directly proportional to the amount of ATP. In the organism and said reaction medium, the extracellular ATP disappears relatively rapidly, either through re-use, or mainly by degradation.

The term “free intracellular ANs” is intended to mean herein the ANs present in the free state in the cell, more specifically in the cytoplasm. The invention does not therefore relate to the non-free ANs that are found in the cell and that are bound to DNA or RNA.

ATP is involved in the cell as a source of energy (mechanical energy, osmotic energy, chemical energy, caloric energy, light energy), phosphate donor, pyrophosphate donor, AMP donor and adenosine donor.

The ATP content in cells from the same species varies greatly depending on the physiological state. The detection threshold is limited in general to 10³ bacteria. A better sensitivity will be attained according to the invention, said sensitivity ranging from 1 attomol of ATP (without stabilization of the light signal emitted) to 0.5 attomol of ATP (with stabilization of said signal), which corresponds approximately to the average content of total free intracellular ANs of a bacterium.

When considering relationship (2), the intracellular content of free cyclic adenosine monophosphate (cAMP), which is the precursor involved in the synthesis of AMP, is ignored here since (i) the intracellular concentration of this product is relatively low and especially (ii) the technique as proposed below involves the conversion of ADP to AMP and then of AMP to ATP, thereby decreasing said cAMP content.

According to the invention, use is made of the well-known principle of the firefly (Photinus pyralis), which functions with an enzyme (luciferase), a luminiferous substrate (luciferin) and a coenzyme (in this case ATP). The result is often displayed on a photometer (or luminometer) in RLU, which, although proportional to the amount of ATP, does not make it possible to determine from one sample to the other the real concentration of ATP.

In order to overcome this difficulty, the introduction of a known amount (for example 10² to 10 pmol of ATP) is recommended after the first reading (carried out without the introduction of ATP). However, the “metered” introduction technique does not allow a quantitative determination of the count since the ATP content in said cells does not remain constant: there is a rapid turnover depending on the physiological state.

On the other hand, the cells of a given species all have the same AN content. According to the invention, by determining the AN content, expressed in the form of ATP, it will be possible to carry out quantitative determinations for counting cells, different than viruses.

Advantageously, step (10) of the method of the invention, which relates to isolation and concentration, is carried out by
membrane filtration,
evaporation-centrifugation, in particular under vacuum and at ambient temperature (15-25° C.), and/or
immunocapture.

The immunocapture technique is preferred. It makes it possible to concentrate and purify the cells by binding the latter by means of immobilized antibodies. In practice, these antibodies can be directed against surface antigens of the cells without destroying said cells. Also in practice, these antibodies are immobilized on beads of magnetic latex for the purpose of concentrating and purifying the cell-antibody-bead type conjugation products in a magnetic field and recovering said conjugation products. As a variant, nonmagnetic or nonmagnetizable beads, attached to the antibodies which bind the cells, also make it possible, by settling out, to concentrate and purify the cells. Also as a variant, the concentration/purification stage can be carried out on an affinity column.

Said conjugation products are then separated, if necessary, in particular by elution, so as to have a concentrated liquid composition of cells which are no longer bound to the antibodies. Where appropriate, in order to limit the dilutions which decrease the sensitivity, it may be judicious to concentrate said liquid composition by means of an evaporation-centrifugation device (operating at from 2000-10 000 revs/15 minutes to 2000-10 000 revs/minute), which makes it possible dry a large number of samples in a few minutes, without any loss of products. Evaporation-centrifugation at ambient temperature offers the advantage of being able to remove most of the water from the medium containing the cells.

Also advantageously, step (2°), relating to the lysis of the cell wall, is carried out in the medium resulting from step (1°) by addition of an aqueous buffer containing
(i) Tris plus EDTA, and/or
(ii) DMSO,

and then (a) treatment in a microwave (for approximately 1 minute) in order to open up the cells, (b) rapid cooling (in particular in a refrigerator) and, if necessary, (c) centrifugation in order to recover the resulting liquid medium.

The lysis is required in order to be able to gain access to the free intracellular ANs, to convert the ADP and AMP to ATP and to bring the ATP resulting from said lysis and/or said conversion into contact with the substrate (luciferin) and the enzyme (luciferase).

As indicated above, step (3°) relating to the conversion of ADP and AMP to ATP is carried out by means of myokinase and pyruvate kinase. The reaction mechanisms are the following:

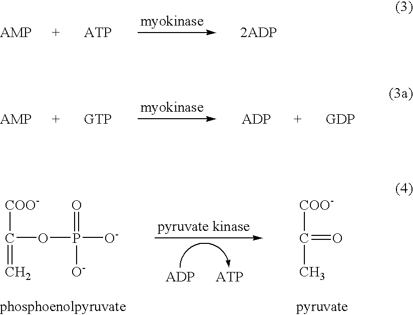

In order to gain time, step (3°) of the method of the invention, relating to the conversion of ADP and AMP to ATP, can be implemented at the same time as step (2°).

Advantageously, step (4°) of the method of the invention is implemented with firefly (Photinus pyralis) luciferin and luciferase. The substrate and the enzyme can be extracted together from the firefly.

In practice, it is recommended to carry out step (5°), relating to the measurement of the light emitted by reaction (1), in the presence of a substance that stabilizes the emission of photons at a value that is substantially constant for at least 10 minutes. Among the substances which are suitable for this purpose, mention may be made of:
pyruvate orthophosphate dikinase (PPDK), which converts the AMP and the pyrophosphate, produced during the abovementioned reaction (1), to ATP, and
adenosine phosphate deaminase, which degrades the residual ADP and/or AMP that may be present in the reaction medium.

The first enzyme provides a stable signal by regenerating ATP in a substantially continuous manner. The second enzyme makes it possible to reduce the background noise due to the residual presence of ADP and/or of AMP, without the process of using ATP as a light energy source being disturbed.

Said second enzyme, adenosine phosphate deaminase, is more advantageously used to eliminate the nucleotide residues in the reaction medium, and more particularly to remove, by destroying them, the extracellular nucleotide residues present, where appropriate, in the sample during the abovementioned step (1°).

In practice, the use of PPDK in step (5°) is more particularly recommended in order to stabilize the emission of photons in accordance with reaction (1).

The method of the invention is particularly suitable for detecting and counting (i) sporulated bacteria, such as anthrax, and (ii) legionellae, salmonellae and other unicellular organisms such as amebae.

Other advantages and characteristics of the invention will be understood more clearly on reading the following implementation examples. Of course, these elements are not limiting, but are provided by way of illustration.

### EXAMPLE 1

### Counting of Anthrax

An aqueous sample is obtained, by sparging, from a sample of 1 L of air containing anthrax strains, to be counted. The strains present are immunocaptured by means of a column comprising immobilized anti-anthrax polyclonal antibodies. The anthrax strains thus purified and concentrated are collected in a small volume of aqueous buffer. Further concentration is carried out by evaporation-concentration under vacuum at ambient temperature. The residues of nucleotides such as ANs, that may be present in the resulting aqueous medium, are removed by means of adenosine phosphate deaminase, which is subsequently inactivated.

The wall of the anthrax strains is lyzed by adding Tris and EDTA and then placing said strains in a microwave. The liquid medium which contains the intracellular ANs is recovered by centrifugation. Myokinase and pyruvate kinase are added in order to convert the AMP and ADP to ATP. The firefly luciferin and the firefly luciferase are added with PPDK in order to stabilize the emission of photons.

The multiplied photons are measured by means of a known device, in RLU. 2 μl of ATP are added and the multiplied photons are remeasured in RLU.

The same procedure is repeated using a known amount (50 strains) of anthrax, and it is determined that the initial liter of air contained 65 strains of anthrax.

### EXAMPLE 2

### Counting of Streptococcus faecalis

The procedure as indicated in example 1 is carried out using the soil from a sheepfold presumed to be infected with Streptococcus faecalis.

It is observed that the soil contains 260 CFU/L of Streptococcus faecalis.

### EXAMPLE 3

### Development of a Protocol for Identifying Legionellae

I—Obtaining the AN/Cell Ratio for Bacteria in Pure Culture

A—Establishment of a Signal Intensity/Number of Legionellae Relationship

The objective is to obtain an average value for the ANs per living Legionella pneumophila cell in order to perform a count per culture and to select the optimal working conditions.

Parameters Studied:

| | |
|---|---|
| Temperature | ambient |
| Buffer conditions | Tris |
| pH | 7.75 |
| Lysis conditions | 100 μl DMSO then 500 μl Tris, pH 7.75 |
| or | 600 μl boiling Tris (microwave for 3 min) |
| Reaction volume | 200 μL of sample with 1 IU of pyruvate |
| | kinase and 1 IU of adenylate kinase + PEP |
| | (time: 10 min) |

Addition of 10 μl of LL (firefly luciferin/luciferase complex)

| | |
|---|---|
| Signal acquisition time | 10 seconds (RLU AN) |
| Addition of 10 μl of ATP | 10 seconds (RLU AN + ATPs) |
| (100 pmol) | |
| After immunoseparation: | the final result is obtained in |
| | less than 15 minutes |

B—Assays on Bacteria Immobilized on Magnetic Beads

Parameters Studied:
Nature of the magnetic bead.
Capture buffer conditions.
Conditions for lysis on microbeads.
Sensitivity study.

Objectives:
to evaluate the signal interferences with magnetic beads of various natures (silica, polystyrene, ferrofluid, etc.),
possible need to separate the microbeads from the legionellae before measurement (elution).

II—Obtaining and Optimizing the Signal on Bacteria Under Natural Conditions

A—Evaluation of the Level of Background Noise of the Samples

Parameters Studied:
Nature of the sample.
Conditions for capture in the sample.
Microbead washing conditions.
Sensitivity study.

Objectives:
to evaluate signal interferences with the nature of the sample: hot wastewater, water from air-cooled cooling towers, river water, deionized water, and
to develop the conditions for washing the beads after capture in order to remove these interferences.

B—Tests on Natural Samples and Optimization

B1—Concentration

The samples of raw water are pre-concentrated (volume of 50 ml to 1 l brought to 1-5 ml). This step makes it possible to increase the sensitivity and to save on the immunoseparation (IMS) means.

The samples used are concentrated either:
by centrifugation,
by filtration,
by magnetic separation with ApoH, and/or
by magnetic separation on silica beads.

In the case of legionellae, it is the latter concentration technique which is preferred since legionellae can be released from binding with the beads, allowing optimized IMS immunocapture.

The concentration and the recovery of the bead-legionellae complexes can be carried out by various techniques known in the art. The legionellae released from the magnetic beads are resuspended in order to be subjected to the optimized IMS protocol as follows:

B2—Optimization of the Protocol After Capture in 1 ml

The parameters involved in the capture are the following:
The capture antibody: anti-Lp1-14 or anti-Lp1.
The IMS buffer.
The antibody load at the surface of the bead.
The bead volume.
The number of washes after capture.
The incubation time.

B3—Application of the Capture Protocol to Volumes Ranging from 1 to 5 ml

The protocol optimized for a volume of 1 ml will be applied to increasing volumes ranging from 1 to 5 ml.

According to the capture yields obtained, it is necessary to adjust certain parameters such as the volume of beads or the incubation time.

B4—Quantification
Once the immunocapture step has been completed, the beads are recovered (by magnetization) with the Legionella pneumophila (dead or living).
The beads are placed in the lysis solution, which may be:
   100 μl of DMSO, which are stirred for 30 seconds and then 500 μl of Tris buffer, pH 7.75, are added,
   600 μl of Tris EDTA buffer, which is brought to boiling for between 1 and 2 minutes in a microwave.
After cooling if necessary, 10 μl of a solution containing the following are added:
   the enzymes for converting AMP and ADP to ATP,
   i.e. PK and AK, at a rate of 1-3 IU,
   the Tris buffer salts (Mg and K),
      phosphoenol pyruvate.
The mixture is incubated for 5 to 10 min at ambient temperature (15-25° C.).
The mixture is distributed into 3 tubes at a rate of 200 μl/tube.
10 μl of the luciferin-luciferase complex (from the company Controlife) are injected.
The tube is introduced into the luminometer.
RLUs=RLUs AN is integrated for 10 seconds.
Immediately after, 10 μl of standard ATP solution [10 to 100 pmol] are injected.
RLUs=RLU AN+ATPs is integrated for 10 seconds.
The amount of picomoles of AN, in ATP equivalents, in the sample is thus calculated.
With the concentration of AN/legionella known, the amount of live legionellae is calculated (the molecular weight of ATP is 551)

The duration of the manipulation, from sampling to the final result, does not exceed 60 minutes for the detection of 10 live legionellae.

III—Overall Determination of a Biomass Related Back to Legionella or E. coli Equivalents

This is a method derived from the previous method, which makes it possible to perform rapid and economical screening. Thus, for example:
(a) when it is desired to verify whether an air-cooled cooling tower has more than 1000 legionellae/l:
   The legionellae are concentrated with the magnetic beads of silica.
   The beads are directly immersed in 100 μl of DMSO and the assay is carried out as previously.
   If the values obtained are below 1000 legionella equivalents, it is not necessary to perform a specific count (gain in time and cost) or to intervene.
   A threshold value for intervention can be set, for example 1500 legionella equivalents.
(b) when it is desired to verify whether water from a swimming area has fewer than 100 E. coli:
   In this case, preference will be given to concentration by means of magnetic beads with the ApoH protein, which, in addition to the fact that its ability to interact with multiple microorganisms is already advantageous, has a characteristic which makes it even more valuable in the medical diagnosis field: it recognizes only pathogens in the infectious state, i.e. undergoing multiplication.
   The procedure will progress in the same manner as previously.

Overall, this approach does not correspond to a specific count. It nevertheless offers the advantage of reducing the amount of time taken for the manipulations and the cost. The advantageous aspect here is that the determination of value is related back to that of an equivalent.

### EXAMPLE 4

### Correlation Between AN and Cells

Using the Arthrobacter NS48 strain, the evolution of the AN/cell ratio as a function of the culture time for this strain was studied according to the protocol established in example 3, the initial bacterial population being 5×10⁶ cells/ml and 3.5×10⁸ cells/ml.

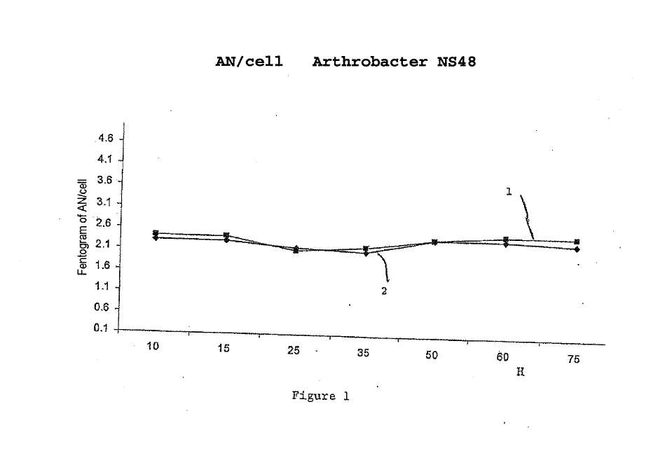

The results obtained are reported in FIG. 1, hereinafter, in the diagram femtogram (1 fg=10⁻¹⁵ g) of AN per bacterial cell (along the y-axis), as a function of the culture time in hours (along the x-axis), in the absence of NaCl (curve 1) and in the presence of 7% (w/v) of NaCl (curve 2). It is noted that the variation in AN is from 2.2 to 2.5 fg per cell between curves 1 and 2: these two curves are virtually equivalent and substantially horizontal.

### EXAMPLE 5

### Comparison of the ATP/Cell and AN/Cell Ratios

The correlation between the ATP/cell ratio was carried out according to said protocol established in example 3 [where the ATP content is variable over time, only the content of total ANs for a given bacterial species is constant in view of the abovementioned equation (2)].

The statistical results obtained are reported in table I which follows. They demonstrate that there is a better correlation for the values of the AN/cell ratio than for the ATP/cell ratio. This correlation is constant in the case of ANs, regardless of the physiological state of the cell and of its environment. It is not necessary to prepare graphs, it is sufficient, as a first approximation, to take into account, for the ANs and the AN/cell ratio, an average value obtained after many cultures of the desired organism.

For example, for E. coli, the average value is 5.27 fg of AN/cell, regardless of the growth phase, whereas, over the same time, the ATP content varies from 0.1 to 1.5 fg/cell.

TABLE I

| Strains | ATP/cell | AN/cell |
|---|---|---|
| Staphylococcus aureus (a)(c) | y = 2.4x + 472 | y = 10.5x + 2062 |
| | r = 1.5x − 473 | r = 0.99 |
| Escherichia coli (b)(c) | y = 1.5x − 473 | y = 6x − 6375 |
| | r = 0.932 | r = 0.987 |
| Pseudomonas aeruginosa (b)(d) | y = 2.4x − 251 | y = 19x − 2015 |
| | r = 0.965 | r = 0.995 |
| Vibrio natriegenes (b)(d) | y = 1.7x + 453 | y = 8x − 508 |
| | r = 0.97 | r = 0.99 |
| Bacillus cereus (b)(d) | y = 2.4 + 1809 | y = 1.3x − 3.5 |
| | r = 0.94 | r = 1 |

Notes
(a) strain from the CHU (University Hospital Center) of Clermont-Ferrand
(b) ATCC strain
(c)

[symbol]

1. The use of bioluminescence according to reaction (1):
luciferin+ATP+O₂+Mg²⁺+luciferase→oxyluciferin+photons, (1)
is provided for detecting and counting living cells of a given species that may be present in a liquid sample, said use being characterized in that it implements measuring the content of total free intracellular adenyl nucleotides (ANs) expressed in the form of ATP, of living cells of a given nonviral species, taking into account the fact that the sum of free intracellular ATP, ADP and AMP of said family is constant according to relationship (2):
[AN]=[ATP]+[ADP]+[AMP]=Ct, (2)
after having converted the free intracellular ADP and AMP to ATP by means of myokinase and pyruvate kinase, said measurement being carried out (i) without the addition of ATP and (ii) after the addition of a known amount of ATP.

2. A method is provided for detecting and counting the cells of a given nonviral species that are liable to be present in a liquid sample (S), in particular bacteria, by means of a method of bioluminescence according to reaction (1):
luciferin+ATP+O₂+Mg²⁺+luciferase→oxyluciferin+photons, (1)
said method, which is based on the fact that, for a living cell of a given nonviral species, the sum of the intracellular adenyl nucleotides (ANs) is constant according to relationship (2):
[AN]=[ATP]+[ADP]+[AMP]=Ct, (2)
being characterized in that it comprises the following steps consisting of:
(1°) isolating and concentrating the cells of said given species that are liable to be present in the sample (S), after having to remove the extracellular ANs that may be contained in said sample;
(2°) lysing the wall of the cells;
(3°) treating the resulting liquid medium in order to convert the intracellular ADP and AMP contained in said liquid medium to ATP;
(4°) introducing, into the medium resulting from step (3°), a luciferin and a luciferase, first (i) without the addition of ATP, and then (ii) after the addition of a known amount of ATP;
(5°) measuring the amplified signal of the light emitted by reaction (1) without the addition of ATP, and then after the addition of a known amount of ATP;
and
(6°) determining the content of total free intracellular ANs in the form of ATP, by comparison with the reproduction of steps (1°) to (5°) with a known population of said cells.

3. The method as claimed in claim 2, in particular for the quantitative detection of a bacterium (B), said method being characterized in that the isolation and concentration step (1°) is carried out by
membrane filtration,
evaporation-centrifugation, in particular under vacuum, and/or
immunocapture.

4. The method as claimed in claim 2, in particular for the quantitative detection of a bacterium (B), said method being characterized in that step (2°) for lysis of the cell wall is carried out in the medium resulting from step (1°) by addition of an aqueous buffer containing
(i) Tris plus EDTA, and/or
(ii) DMSO,
and then (a) treatment in a microwave in order to open up the cells, (b) rapid cooling and, if necessary, (c) centrifugation in order to recover the resulting liquid medium.

5. The method as claimed in claim 2, in particular for the quantitative detection of a bacterium (B), said method being characterized in that step (3°) for conversion of the ADP and AMP to ATP is carried out by means of myokinase and pyruvate kinase.

6. The method as claimed in claim 2, in particular for the quantitative detection of a bacterium (B), said method being characterized in that step (3°) is implemented at the same time as step (2°).

7. The method as claimed in claim 2, in particular for the quantitative detection of a bacterium (B), said method being characterized in that: step (4°) is implemented with firefly (Photinus pyralis) luciferin and luciferase.

8. The method as claimed in claim 2, in particular for the quantitative detection of a bacterium (B), said method being characterized in that step (5°) for measuring the light emitted by reaction (1) is carried out in the presence of a substance that stabilizes the emission of photons at a value that is substantially constant for at least 10 minutes.

9. The method as claimed in claim 2, in particular for the quantitative detection of a bacterium (B), said method being characterized in that: step (6°) is implemented by comparison with a system of graphs established from several known populations of said cells and their contents of total free intracellular ANs expressed in the form of ATP.

10. The method as claimed in claim 2, characterized in that the sensitivity threshold is from 0.5 to 1 attomol of ATP.

11. The use of the method as claimed in claim 2, for counting bacteria in the form of spores.

12. An assay kit for implementing the method as claimed in claim 2, characterized in that it comprises the firefly luciferin/luciferase combination, ATP for the metered addition, myokinase, pyruvate kinase and, where appropriate, pyruvate orthophosphate dikinase.
